(19) United States
(12) Patent Application Publication
(10) Pub. No.: US 2026/0212959 A1
(43) Pub. Date: Jul. 23, 2026

(54) DNA STORAGE DATA RECONSTRUCTION METHOD AND SYSTEM BASED ON NON-REDUNDANT DE BRUIJN GRAPH

(71) Applicant: DALIAN UNIVERSITY, DALIAN (CN)

(72) Inventors: QIANG ZHANG, DALIAN (CN); BIN WANG, DALIAN (CN); YUNZHU ZHAO, DALIAN (CN); XIAOPENG WEI, DALIAN (CN); SHIHUA ZHOU, DALIAN (CN); HUI LV, DALIAN (CN); HUIZI MAN, DALIAN (CN); WEI JIANG, DALIAN (CN)

(73) Assignee: DALIAN UNIVERSITY, DALIAN (CN)

(21) Appl. No.: 19/034,623

(22) Filed: Jan. 23, 2025

Related U.S. Application Data
(63) Continuation-in-part of application No. PCT/CN2024/144062, filed on Dec. 31, 2024.

(51) Int. Cl. G16B 50/30; G16B 30/10; G16B 40/00

(52) U.S. Cl. CPC G16B 50/30; G16B 30/10; G16B 40/00

(57) ABSTRACT

This application discloses a method and system for DNA storage data reconstruction based on a non-redundant de Bruijn graph. The method includes the following steps: grouping DNA sequences with a Levenshtein distance less than or equal to a set threshold into the same clustering subgraph; connecting nodes with the smallest Levenshtein distance between DNA sequences, where the node with the highest degree represents the backbone sequence of the clustering subgraph; using the backbone sequence as a template sequence for sequencing data alignment to construct a beam search graph; performing error correction and selecting the optimal path using a beam search algorithm to obtain a consensus sequence; constructing a non-redundant De Bruijn graph; deleting nodes connected by edges with weights below a set threshold; selecting paths based on node information in the non-redundant De Bruijn graph and the consensus sequence; and sequence obtained after path selection is the reconstructed sequence.

### TECHNICAL FIELD

The present application relates to the field of DNA storage technology, and specifically to a method and system for DNA storage data reconstruction based on a non-redundant De Bruijn graph.

### BACKGROUND

DNA storage involves encoding data into DNA sequences, writing the data through synthesis, reading the DNA sequences using high-throughput sequencing technology, and decoding the sequences to retrieve the data. In some cases, the key steps in DNA storage are data writing and reading. During the data writing phase, the core task is to map binary data to DNA sequences based on efficient encoding rules. During the data reading phase, the original data is fully recovered by decoding the amplified and sequenced DNA sequences. However, due to inherent limitations in biotechnology, unavoidable errors may occur during DNA storage, leading to data corruption and loss. Therefore, the challenges in DNA storage not only include how to write data into DNA but also how to efficiently reconstruct the original data from DNA sequences.

### SUMMARY

The objective of the present application is to provide a method and system for DNA storage data reconstruction based on a non-redundant De Bruijn graph, which controls the redundancy of stored information at a relatively low level and ensures data recovery rates even under high error rates, thereby effectively reconstructing the data.

To achieve the above objective, the present application provides the following solutions:

According to a first aspect of the embodiments of the present disclosure, a method for DNA storage data reconstruction based on a non-redundant De Bruijn graph is provided, comprising the following steps: obtaining the Levenshtein distance between DNA sequences, grouping DNA sequences with a Levenshtein distance less than or equal to a set threshold into the same clustering subgraph, thereby dividing multiple DNA sequences into multiple clustering subgraphs; treating each DNA sequence in the clustering subgraph as a node, connecting nodes with the smallest Levenshtein distance between DNA sequences, and identifying the node with the highest degree as the backbone sequence of the clustering subgraph; using the backbone sequence as a template sequence for sequencing data alignment, and constructing a beam search graph based on the relative positions of bases in the backbone sequence for other DNA sequences; performing error correction and selecting the optimal path in the beam search graph using a beam search algorithm, and performing multiple sequence alignment on the DNA sequences involved in the optimal path to obtain a consensus sequence; segmenting the DNA sequences of each clustering subgraph into k-mer sub-sequences and constructing a non-redundant De Bruijn graph using a sliding window, where nodes with k−1 identical bases are connected, and the frequency between nodes is recorded as the weight of the edge; deleting nodes connected by edges with weights below a set threshold to reduce the number of judgments in subsequent path selection; selecting paths based on node information in the non-redundant De Bruijn graph and the consensus sequence; the sequence after path selection is the reconstructed sequence.

According to a second aspect of the embodiments of the present disclosure, a system for DNA storage data reconstruction based on a non-redundant De Bruijn graph is provided, comprising: a clustering subgraph acquisition module, which obtains the Levenshtein distance between DNA sequences and groups DNA sequences with a Levenshtein distance less than or equal to a set threshold into the same clustering subgraph, thereby dividing multiple DNA sequences into multiple clustering subgraphs; a backbone sequence acquisition module, which treats each DNA sequence in the clustering subgraph as a node, connects nodes with the smallest Levenshtein distance between DNA sequences, and identifies the node with the highest degree as the backbone sequence of the clustering subgraph; a beam search graph acquisition module, which uses the backbone sequence as a template sequence for sequencing data alignment and constructs a beam search graph based on the relative positions of bases in the backbone sequence for other DNA sequences; a consensus sequence acquisition module, which performs error correction and selects the optimal path in the beam search graph using a beam search algorithm, and performs multiple sequence alignment on the sequences involved in the optimal path to obtain a consensus sequence; a non-redundant De Bruijn graph acquisition module, which segments the DNA sequences of each clustering subgraph into k-mer sub-sequences and constructs a non-redundant De Bruijn graph using a sliding window, where nodes with k−1 identical bases are connected, and the frequency between nodes is recorded as the weight of the edge; a deletion module, which deletes nodes connected by edges with weights below a set threshold to reduce the number of judgments in subsequent path selection; a path selection module, which selects paths based on node information in the non-redundant De Bruijn graph and the consensus sequence; and a reconstruction module, where the sequence after path selection is the reconstructed sequence.

According to a third aspect of the embodiments of the present disclosure, an electronic device is provided, comprising a memory, a processor, and a computer program stored on the memory and executable by the processor, wherein the processor executes the program to implement the method for DNA storage data reconstruction based on a non-redundant De Bruijn graph.

According to a fourth aspect of the embodiments of the present disclosure, a non-transitory computer-readable storage medium is provided, having a computer program stored thereon, wherein the program, when executed by a processor, implements the method for DNA storage data reconstruction based on a non-redundant De Bruijn graph.

### BRIEF DESCRIPTION OF THE DRAWINGS

To more clearly illustrate the embodiments of the present application or the technical solutions in the prior art, the following will briefly describe the drawings used in the embodiments. It is obvious that the drawings in the following description are only some embodiments of the present application. For those skilled in the art, other drawings can be obtained based on these drawings without creative effort.

FIG. 1 is a flowchart of a method for DNA storage data reconstruction based on a non-redundant De Bruijn graph according to one or more embodiments.

FIG. 2 is a schematic diagram comparing the reconstruction rates of BSDBG and DBGPS under mixed error rates according to one or more embodiments.

FIG. 3 is a schematic diagram comparing the data recovery rates of BSDBG and DBGPS under mixed error rates according to one or more embodiments.

### DETAILED DESCRIPTION

The technical solutions in the embodiments of the present application will be clearly and completely described below with reference to the accompanying drawings. It is obvious that the described embodiments are only a part of the embodiments of the present application, and not all of them. Based on the embodiments in the present application, all other embodiments obtained by those skilled in the art without creative effort shall fall within the scope of protection of the present application.

It should be noted that the following detailed description is exemplary and is intended to provide further explanation of the present application. Unless otherwise specified, all technical and scientific terms used herein have the same meanings as commonly understood by those skilled in the technical field of the present application.

It should be noted that the terminology used herein is for the purpose of describing specific embodiments and is not intended to limit the exemplary embodiments of the present application. As used herein, unless the context clearly indicates otherwise, the singular forms are intended to include the plural forms, and it should also be understood that when the terms “comprise” and/or “include” are used in this specification, they specify the presence of features, steps, operations, devices, components, and/or their combinations.

It should be noted that the flowcharts and block diagrams in the drawings illustrate the possible architecture, functions, and operations of the methods and systems according to various embodiments of the present disclosure. It should be noted that each block in the flowcharts or block diagrams may represent a module, a program segment, or a part of code, which includes one or more executable instructions for implementing the specified logical functions. It should also be noted that in some alternative implementations, the functions noted in the blocks may occur in a different order than the order noted in the drawings. For example, two blocks shown in succession may actually be executed substantially concurrently, or they may sometimes be executed in the reverse order, depending on the functions involved. It should also be noted that each block in the flowcharts and/or block diagrams, and combinations of blocks in the flowcharts and/or block diagrams, may be implemented by a dedicated hardware-based system that performs the specified functions or operations, or by a combination of dedicated hardware and computer instructions.

In one embodiment, the present embodiment provides a method for DNA storage data reconstruction based on a non-redundant De Bruijn graph, comprising the following steps:

Step 1: Obtain the Levenshtein distance between DNA sequences, and group DNA sequences with a Levenshtein distance less than or equal to a set threshold into the same clustering subgraph, thereby dividing multiple DNA sequences into multiple clustering subgraphs.

For example, assume the threshold is 3, and dist(L1, L2) represents the Levenshtein distance between sequence L1 and sequence L2, where sequence L1 is ATGGCCATTGTA, sequence L2 is ATGGCTATTGTA, sequence L3 is ATGGCCATTGCA, and sequence L4 is ATGGCAATTGTA. Since dist(L1, L2)=1, dist(L1, L3)=1, dist(L1, L4)=2, dist(L2, L3)=2, dist(L2, L4)=3, and dist(L3, L4)=3, all fall within the threshold range of 3, they are grouped into the same clustering subgraph.

In one embodiment, the threshold is dynamically adjustable. For example, based on the noise level of the dataset, the threshold for the Levenshtein distance is dynamically adjusted. For high-noise data, the threshold is appropriately increased to capture more similar sequences; for low-noise data, the threshold is decreased to improve clustering accuracy.

In one embodiment, hierarchical clustering operations may be performed, where after initial clustering, each clustering subgraph is further subdivided to form a hierarchical clustering structure.

In one embodiment, the calculation of the Levenshtein distance may be parallelized, for example, using GPU acceleration or multi-threading technology to accelerate the calculation process.

Step 2: Treat each DNA sequence in the clustering subgraph as a node, connect nodes with the smallest Levenshtein distance between DNA sequences, and identify the node with the highest degree as the backbone sequence of the clustering subgraph.

For example, the distance between sequence L1 and sequence L2 is the smallest at 1, so sequence L1 and sequence L2 are connected first. The distance between sequence L1 and sequence L3 is also 1, so sequence L1 and sequence L3 are connected. The distance between sequence L1 and sequence L4 is 2, but since L1 is already connected to L2 and L3, and the distance between L4 and L2 is 3, the connection between L1 and L4 may be optional. Since node L1 has the highest degree (with 3 edges), L1 can be considered the backbone sequence of this clustering subgraph. That is, the process of selecting a node as the backbone sequence takes into account indicators such as sequence length and coverage.

Step 3: Use the backbone sequence as a template sequence for sequencing data alignment, and construct a beam search graph for other DNA sequences based on the relative positions of bases in the backbone sequence.

In one implementation: each base in the backbone sequence is treated as a node, and adjacent bases are connected as adjacent nodes, with the weight set to 1; gradually add adjacent nodes represented by the bases of the node with the highest degree, and match the adjacent nodes based on their positions in the backbone sequence, incrementally increasing the weight of the edges of the matched adjacent nodes by 1. It should be noted that when constructing the beam search graph for other DNA sequences based on the relative positions of bases in the backbone sequence, three types of errors may occur: substitution errors, deletion errors, and insertion errors; different error types are represented by the following formulas:
For a substituted node v′_{re_i}: w(vᵢ₋₁,v′_{re_i})+=1 and w(v′_{re_i},vᵢ₊₁,)+=1.
For a deleted node v′_{de_i}: w(vᵢ₋₁,vᵢ₊₁)+=1.
For an inserted node vᵢ′_{ns_i}: w(vᵢ₋₁,vᵢ′_{ns_i})+=1 and w(vᵢ′_{ns_i},vᵢ)+=1.
For any k≠i, if vₖ and vₖ₊₁ have no errors: w(vₖ,vₖ₊₁)+=1.

Where i represents the index of the error position, vᵢ represents the node corresponding to index i in the original sequence, w(vᵢ₋₁, v′_{re_i}) represents the weight from node vᵢ₋₁ to node v′_{re_i}, w(v′_{re_i}, vᵢ₊₁) represents the weight from node v′_{re_i} to node vᵢ₊₁, w(vᵢ₋₁, vᵢ₊₁) represents the weight from node vᵢ₋₁ to node vᵢ₊₁, w(vᵢ₋₁, vᵢ′_{ns_i}) represents the weight from node vᵢ₋₁ to node vi′_{ns_i}, (vi′_{ns_i}, vᵢ) represents the weight from node vi′_{ns_i} to node vᵢ, and w(vₖ, vₖ₊₁) represents the weight from node vₖ to node vₖ₊₁. Additionally, the original sequence corresponds to sequences L1, L2, L3, and L4 mentioned earlier.

In one embodiment, Step 3 further includes dynamically updating the beam search graph: dynamically adjusting the edge weights based on the results of error correction.

Step 4: Perform error correction and select the optimal path in the beam search graph using a beam search algorithm, and perform multiple sequence alignment on the DNA sequences involved in the optimal path to obtain a consensus sequence.

In the beam search algorithm, B is called the beam width. When B is 1, BS is equivalent to best-first search; when applying BS to the beam search graph, starting from a specified starting node, initialize B empty paths as the output set Y=[y1, y2, . . . , yB], where y1 is the first path, y2 is the second path, and yB is the B-th path; in each step, extend each path by adding a base; for each node vx in the beam search graph, obtain the extension path score S(vx|yb) for all paths yb that have vx as a successor node: S(vx|yb)=W(yb)+w(ybt−1, vx).

Where W(y_{b}) represents the path weight of path

$${y}_{b} ; w ( {y}_{b}^{t - 1} , {v}_{x} )$$

represents the weight of the edge from y_{b}^{t-1} to node vₓ, and y_{b}^{t-1} represents the (t−1)-th base in path b.

Through the above path generation method, multiple candidate paths are generated in the beam search graph, each representing a preliminary corrected sequence, which helps improve the accuracy of multiple sequence alignment. Finally, the multiple candidate paths are error-corrected using a multiple sequence alignment tool to obtain the consensus sequence.

In one embodiment, Step 4 further includes adaptively adjusting the beam width: dynamically adjusting the beam width (B) based on data complexity and computational resources.

Step 5: Segment the DNA sequences of each clustering subgraph into k-mer sub-sequences and construct a non-redundant De Bruijn graph using a sliding window, where nodes with k−1 identical bases are connected, and the frequency between nodes is recorded as the weight of the edge.

In a specific application example, a fixed k value is selected, representing the length of each sub-sequence. First, starting from the beginning of the DNA sequence, extract a sub-sequence of length k, i.e., a k-mer; then slide with a fixed step size of 1 and repeat this process until the entire DNA sequence is covered; where each node in the non-redundant De Bruijn graph represents a k-mer, and each edge in the non-redundant De Bruijn graph represents the relationship between two adjacent k-mers; nodes with k−1 identical bases are connected, and the frequency between nodes is recorded as the weight of the edge.

In one embodiment, Step 5 further includes dynamically adjusting the k-mer length and the edge weight threshold to adapt to different datasets.

Step 6: Delete nodes connected by edges with weights below a set threshold to reduce the number of judgments in subsequent path selection. By setting an appropriate threshold, k-mers with low frequency that may mislead the assembly process are filtered out, thereby improving the accuracy of assembly.

In one embodiment, Step 6 further includes dynamically adjusting the edge weight threshold based on the coverage and error rate of the dataset.

Step 7: Select paths based on node information in the non-redundant De Bruijn graph and the consensus sequence.

In a specific application example, if the current node has only one successor node, directly select that successor node as the next node; if the current node has multiple successor nodes, consider the position of the k-mer in the consensus sequence and the frequency of the successor nodes to select the next node: based on the position of the current node, find the corresponding k-mer in the consensus sequence, and check if the k-mer of the current node matches the k-mer in the consensus sequence; if they match, select the node corresponding to the next k-mer in the consensus sequence as the successor node; if they do not match, select the node with the highest edge weight as the successor node.

In one embodiment, Step 7 further includes generating a new consensus sequence based on the current path selection results and re-guiding subsequent path selection.

Step 8: The sequence obtained after path selection is the reconstructed sequence.

Based on the path selection results, the k-mers are concatenated into a complete DNA sequence, i.e., the reconstructed sequence. The method supports the processing of mixed sequencing data (such as Illumina, PacBio, Nanopore), combining the advantages of short-read and long-read sequencing to improve the completeness and accuracy of reconstruction.

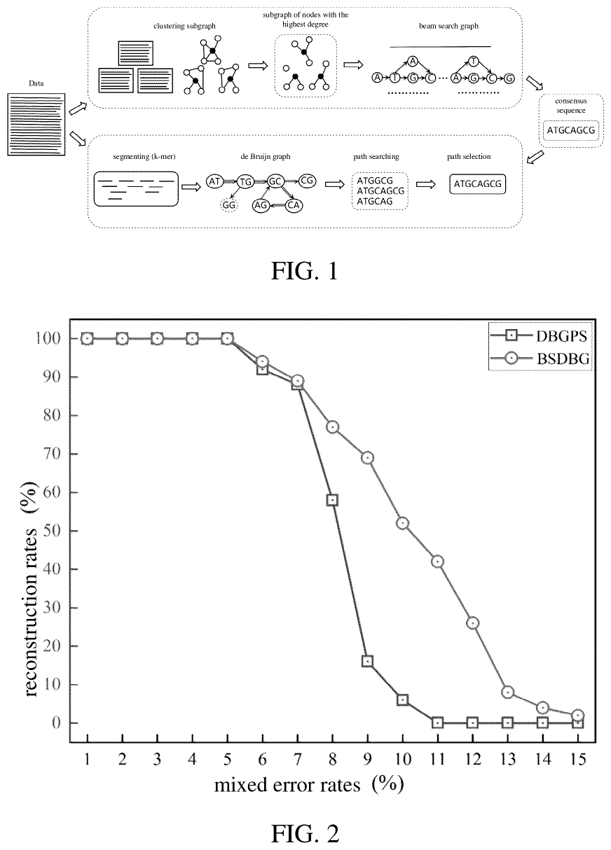

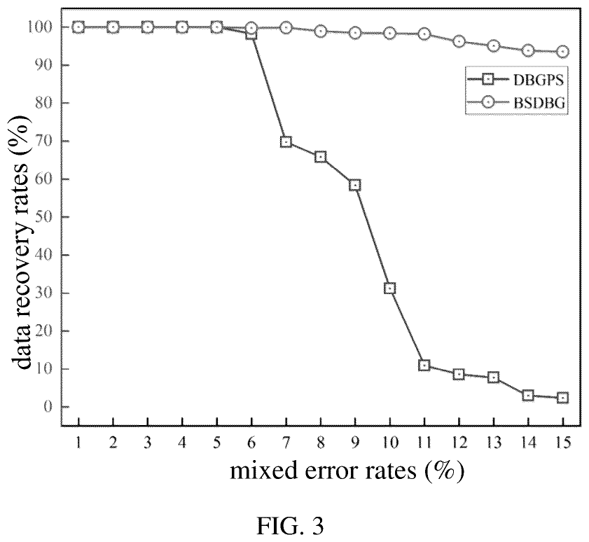

As shown in FIGS. 2 and 3, experimental results demonstrate that the method of this embodiment outperforms other algorithms (such as DBGPS), where BSDBG represents the experimental results of this application. The method forms a complete DNA storage data reconstruction process through steps such as Levenshtein distance-based clustering, backbone sequence selection, beam search graph construction, and non-redundant De Bruijn graph optimization. Its innovation lies in dynamically adjusting parameters, combining multiple sequence alignment and consensus sequence-guided path selection, significantly improving reconstruction accuracy and efficiency. The method is suitable for high-noise DNA data and can be further expanded in application scope through modular design and distributed computing.

Based on the same inventive concept, the present application also provides a system for DNA storage data reconstruction based on a non-redundant De Bruijn graph. The system provides solutions to problems similar to those described in the method above, and thus the specific limitations in one or more system embodiments provided below can be referred to the limitations described in the method above, which will not be repeated here. The embodiment provides a system for DNA storage data reconstruction based on a non-redundant De Bruijn graph, comprising:

A clustering subgraph acquisition module, which obtains the Levenshtein distance between DNA sequences and groups DNA sequences with a Levenshtein distance less than or equal to a set threshold into the same clustering subgraph, thereby dividing multiple DNA sequences into multiple clustering subgraphs.

A backbone sequence acquisition module, which treats each DNA sequence in the clustering subgraph as a node, connects nodes with the smallest Levenshtein distance between DNA sequences, and identifies the node with the highest degree as the backbone sequence of the clustering subgraph.

A beam search graph acquisition module, which uses the backbone sequence as a template sequence for sequencing data alignment and constructs a beam search graph based on the relative positions of bases in the backbone sequence for other DNA sequences.

A consensus sequence acquisition module, which performs error correction and selects the optimal path in the beam search graph using a beam search algorithm, and performs multiple sequence alignment on the sequences involved in the optimal path to obtain a consensus sequence.

A non-redundant De Bruijn graph acquisition module, which segments the DNA sequences of each clustering subgraph into k-mer sub-sequences and constructs a non-redundant De Bruijn graph using a sliding window, where nodes with k−1 identical bases are connected, and the frequency between nodes is recorded as the weight of the edge.

A deletion module, which deletes nodes connected by edges with weights below a set threshold to reduce the number of judgments in subsequent path selection.

A path selection module, which selects paths based on node information in the non-redundant De Bruijn graph and the consensus sequence.

A reconstruction module, where the sequence after path selection is the reconstructed sequence.

In an exemplary embodiment, a computer device is provided, which may be a server or a terminal. The computer device includes a processor, a memory, an input/output interface (I/O), and a communication interface. The processor, memory, and input/output interface are connected via a system bus, and the communication interface is connected to the system bus via the input/output interface. The processor of the computer device provides computing and control capabilities. The memory of the computer device includes a non-volatile storage medium and an internal memory. The non-volatile storage medium stores an operating system, computer programs, and a database. The internal memory provides an environment for the operation of the operating system and computer programs in the non-volatile storage medium. The input/output interface of the computer device is used for information exchange between the processor and external devices. The communication interface of the computer device is used for network connection and communication with external terminals.

In an exemplary embodiment, an electronic device is provided, comprising a memory, a processor, and a computer program stored on the memory and executable by the processor, wherein the processor executes the program to implement the method for DNA storage data reconstruction based on a non-redundant De Bruijn graph, including:

Obtaining the Levenshtein distance between DNA sequences, and grouping DNA sequences with a Levenshtein distance less than or equal to a set threshold into the same clustering subgraph, thereby dividing multiple DNA sequences into multiple clustering subgraphs; treating each DNA sequence in the clustering subgraph as a node, connecting nodes with the smallest Levenshtein distance between DNA sequences, and identifying the node with the highest degree as the backbone sequence of the clustering subgraph; using the backbone sequence as a template sequence for sequencing data alignment, and constructing a beam search graph based on the relative positions of bases in the backbone sequence for other DNA sequences; performing error correction and selecting the optimal path in the beam search graph using a beam search algorithm, and performing multiple sequence alignment on the DNA sequences involved in the optimal path to obtain a consensus sequence; segmenting the DNA sequences of each clustering subgraph into k-mer sub-sequences and constructing a non-redundant De Bruijn graph using a sliding window, where nodes with k−1 identical bases are connected, and the frequency between nodes is recorded as the weight of the edge; deleting nodes connected by edges with weights below a set threshold to reduce the number of judgments in subsequent path selection; selecting paths based on node information in the non-redundant De Bruijn graph and the consensus sequence; the sequence after path selection is the reconstructed sequence.

In an exemplary embodiment, a non-transitory computer-readable storage medium is provided, having a computer program stored thereon, wherein the program, when executed by a processor, implements the method for DNA storage data reconstruction based on a non-redundant De Bruijn graph, including:

Obtaining the Levenshtein distance between DNA sequences, and grouping DNA sequences with a Levenshtein distance less than or equal to a set threshold into the same clustering subgraph, thereby dividing multiple DNA sequences into multiple clustering subgraphs; treating each DNA sequence in the clustering subgraph as a node, connecting nodes with the smallest Levenshtein distance between DNA sequences, and identifying the node with the highest degree as the backbone sequence of the clustering subgraph; using the backbone sequence as a template sequence for sequencing data alignment, and constructing a beam search graph based on the relative positions of bases in the backbone sequence for other DNA sequences; performing error correction and selecting the optimal path in the beam search graph using a beam search algorithm, and performing multiple sequence alignment on the DNA sequences involved in the optimal path to obtain a consensus sequence; segmenting the DNA sequences of each clustering subgraph into k-mer sub-sequences and constructing a non-redundant De Bruijn graph using a sliding window, where nodes with k−1 identical bases are connected, and the frequency between nodes is recorded as the weight of the edge; deleting nodes connected by edges with weights below a set threshold to reduce the number of judgments in subsequent path selection; selecting paths based on node information in the non-redundant De Bruijn graph and the consensus sequence; the sequence after path selection is the reconstructed sequence.

The present application adopts optimization strategies in clustering preprocessing, multiple sequence alignment, and path selection, controlling the redundancy of stored information at a relatively low level, ensuring high data recovery rates even under high error rates, and effectively reconstructing data.

According to the specific embodiments provided by the present application, the present application has the following technical effects:

By grouping DNA sequences with high similarity into the same clustering subgraph, nodes weakly associated with the backbone sequence are filtered out; through the beam search graph, base alignment and preliminary correction of sequences are achieved, high-accuracy candidate paths are identified, and a high-accuracy consensus sequence is obtained through multiple sequence alignment.

The possibility of the same k-mer appearing in the same non-redundant De Bruijn graph from different clusters is reduced, and path selection ambiguity is resolved using the consensus sequence, ensuring the accuracy of assembly results.

Without adding redundant check bits, various errors can be accurately corrected, and original data can be recovered, achieving accurate sequence reconstruction.

It should be noted that the user information (including but not limited to user device information, user personal information, etc.) and data (including but not limited to data used for analysis, stored data, displayed data, etc.) involved in this application are all authorized by the user or fully authorized by all parties, and the collection, use, and processing of relevant data need to comply with relevant regulations.

Those skilled in the art can understand that all or part of the processes in the methods of the above embodiments can be implemented by instructing relevant hardware through a computer program. The computer program can be stored in a non-volatile computer-readable storage medium, and when the program is executed, it may include the processes of the embodiments of the above methods. Among them, any reference to memory, database, or other media in the embodiments provided by this application may include at least one of non-volatile and volatile memory. Non-volatile memory may include read-only memory (ROM), magnetic tape, floppy disks, flash memory, optical memory, high-density embedded non-volatile memory, resistive random-access memory (ReRAM), magnetoresistive random-access memory (MRAM), ferroelectric random-access memory (FRAM), phase-change memory (PCM), graphene memory, etc. Volatile memory may include random-access memory (RAM) or external cache memory. As an illustration and not a limitation, RAM may take various forms, such as static random-access memory (SRAM) or dynamic random-access memory (DRAM).

The databases involved in the embodiments provided by this application may include at least one of relational databases and non-relational databases. Non-relational databases may include blockchain-based distributed databases, etc., but are not limited to this. The processors involved in the embodiments provided by this application may be general-purpose processors, central processing units, graphics processing units, digital signal processors, programmable logic devices, quantum computing-based data processing logic devices, etc., but are not limited to this.

The technical features of the above embodiments can be combined arbitrarily. For the sake of brevity, not all possible combinations of the technical features in the above embodiments are described. However, as long as there is no contradiction in the combination of these technical features, they should be considered within the scope of this specification.

This article uses specific examples to explain the principles and implementation methods of this application. The descriptions of the above embodiments are only used to help understand the methods and core ideas of this application. At the same time, for those skilled in the art, according to the ideas of this application, there will be changes in the specific implementation methods and application scope. In summary, the content of this specification should not be construed as limiting this application.

1. A method for DNA storage data reconstruction based on a non-redundant de Bruijn graph, comprising:
obtaining Levenshtein distances between DNA sequences, and group DNA sequences with a Levenshtein distance less than or equal to a set threshold into the same clustering subgraph, thereby dividing multiple DNA sequences into multiple clustering subgraphs;
treating each DNA sequence in the clustering subgraph as a node, connecting nodes with the smallest Levenshtein distance between DNA sequences, and identifying the node with the highest degree as the backbone sequence of the clustering subgraph;
using the backbone sequence as a template sequence for sequencing data alignment, and constructing a beam search graph for other DNA sequences based on the relative positions of bases in the backbone sequence;
performing error correction and selecting an optimal path in the beam search graph using a beam search algorithm, and performing multiple sequence alignment on the DNA sequences involved in the optimal path to obtain a consensus sequence;
segmenting the DNA sequences of each clustering subgraph into k-mer sub-sequences and constructing a non-redundant de Bruijn graph using a sliding window, where nodes with k−1 identical bases are connected, and the frequency between nodes is recorded as the weight of the edge;
deleting nodes connected by edges with weights below a set threshold to reduce the number of judgments in subsequent path selection;
selecting paths based on node information in the non-redundant de Bruijn graph and the consensus sequence; and
recording obtained sequences after path selection as reconstructed sequences.

2. The method according to claim 1, wherein using the backbone sequence as a template sequence for sequencing data alignment comprises:
treating each base in the backbone sequence as a node, connecting adjacent bases as adjacent nodes, and setting the weight to 1; and
gradually adding adjacent nodes represented by the bases of the node with the highest degree, matching the adjacent nodes based on their positions in the backbone sequence, and incrementally increasing the weight of the edges of the matched adjacent nodes by 1.

3. The method according to claim 1, wherein constructing the beam search graph for other DNA sequences based on the relative positions of bases in the backbone sequence involves three types of errors: substitution errors, deletion errors, and insertion errors; different error types are represented by the following formulas:
for a substituted node v′_{re_i}: w(vᵢ₋₁, v′_{re_i})+=1 and w(v′_{re_i}, vᵢ₊₁)+=1;
for a deleted node v′_{de_i}: w(vᵢ₋₁, vᵢ₊₁)+=1;
for an inserted node vᵢ′_{ns_i}: w(vᵢ₋₁, vᵢ′_{ns_i})+=1 and w(vᵢ′_{ns_i}, vᵢ)+=1;
for any k≠i, if nodes vₖ and vₖ₊₁ have no errors: w(vₖ, vₖ₊₁)+=1;
where i represents the index of the error position, vᵢ represents the node corresponding to index i in the original sequence, w(vᵢ₋₁, v′_{re_i}) represents the weight from node vᵢ₋₁ to node v′_{re_i}, w(v′_{re_i}, vᵢ₊₁) represents the weight from node v′_{re_i} to node vᵢ₊₁, w(vᵢ₋₁, vᵢ₊₁) represents the weight from node vᵢ₋₁ to node vᵢ₊₁, w(vᵢ₋₁, vᵢ′_{ns_i}) represents the weight from node vᵢ₋₁ to node vᵢ′_{ns_i}, (vᵢ′_{ns_i}, vᵢ) represents the weight from node vᵢ′_{ns_i} to node vᵢ, w(vₖ, vₖ₊₁) represents the weight from node vₖ to node vₖ₊₁.

4. The method according to claim 1, wherein in the beam search algorithm:
starting from a specified starting node, initialize B empty paths as an output set Y=[y1, y2, . . . , yB], where y1 is the first path, y2 is the second path, and y_{B} is the B-th path;
in each step, extend each path by adding a base; and
for each node vₓ in the beam search graph, obtain the extension path score S(vₓ|y_{b}) for all paths y_{b} that have vₓ as a successor node.

5. The method according to claim 4, wherein the extension path score S(vx|yb) is calculated as:
$$S ( vx \vert yb ) = W ( y b ) + w ( ybt - 1 , vx ) ;$$
where: W(yb) represents the path weight of path yb, w(ybt−1, vx) represents the weight of the edge from ybt−1 to node vx, ybt−1 represents the (t−1)-th base in path b.

6. The method according to claim 1, wherein segmenting the DNA sequences of each clustering subgraph into k-mer sub-sequences and constructing a non-redundant de Bruijn graph using a sliding window comprises:
selecting a fixed k value representing the length of each sub-sequence;
starting from the beginning of the DNA sequence, extracting a sub-sequence of length k to obtain a k-mer; and
sliding with a fixed step size of 1 and repeat the process until the entire DNA sequence is covered;
wherein in the non-redundant de Bruijn graph, each node represents a k-mer, and each edge represents the relationship between two adjacent k-mers, nodes with k−1 identical bases are connected, and a frequency between nodes is recorded as the weight of the edge.

7. The method according to claim 1, wherein selecting paths based on node information in the non-redundant de Bruijn Graph and the consensus sequence comprises:
if the current node has only one successor node, selecting that successor node as the next node; and
if the current node has multiple successor nodes, considering the position of the k-mer in the consensus sequence and the frequency of the successor nodes to select the next node.

8. The method according to claim 7, wherein if the current node has multiple successor nodes:
based on the position of the current node, finding the corresponding k-mer in the consensus sequence; and
if the k-mer of the current node matches the k-mer in the consensus sequence, then selecting the node corresponding to the next k-mer in the consensus sequence as the successor node, else selecting the node with the highest edge weight as the successor node.

9. The method according to claim 1, further comprising:
dynamically adjusting the threshold based on the noise level of the dataset to improve clustering accuracy.

10. The method according to claim 9, wherein adjusting the threshold based on the noise level of the dataset comprises:
increasing the threshold for data having a noisy level greater than a predetermined value and decreasing the threshold for data having a noisy level less than the predetermined value.

11. The method according to claim 10, further comprising:
after initial clustering, subdividing each clustering subgraph to form a hierarchical clustering structure.

12. The method according to claim 11, wherein the calculation of the Levenshtein distance is parallelized using GPU acceleration or multi-threading technology to accelerate the calculation process.

13. The method according to claim 12, wherein the process of selecting a node as the backbone sequence takes into account indicators such as sequence length and coverage.

14. The method according to claim 13, further comprising:
dynamically adjusting the edge weights based on the results of error correction.

15. The method according to claim 14, further comprising:
dynamically adjusting the beam width (B) based on data complexity and computational resources.

16. The method according to claim 15, further comprising:
dynamically adjusting the k-mer length and the edge weight threshold to adapt to different datasets.

17. The method according to claim 16, further comprising:
dynamically adjusting the edge weight threshold based on the coverage and error rate of the dataset.

18. The method according to claim 17, further comprising:
generating a new consensus sequence based on the current path selection results and re-guiding subsequent path selection.

19. An electronic device, comprising:
a memory,
a processor, and
a computer program stored on the memory and executable by the processor, wherein the processor executes the program to implement the method comprising:
obtaining Levenshtein distances between DNA sequences, and group DNA sequences with a Levenshtein distance less than or equal to a set threshold into the same clustering subgraph, thereby dividing multiple DNA sequences into multiple clustering subgraphs;
treating each DNA sequence in the clustering subgraph as a node, connecting nodes with the smallest Levenshtein distance between DNA sequences, and identifying the node with the highest degree as the backbone sequence of the clustering subgraph;
using the backbone sequence as a template sequence for sequencing data alignment, and constructing a beam search graph for other DNA sequences based on the relative positions of bases in the backbone sequence;
performing error correction and selecting an optimal path in the beam search graph using a beam search algorithm, and performing multiple sequence alignment on the DNA sequences involved in the optimal path to obtain a consensus sequence;
segmenting the DNA sequences of each clustering subgraph into k-mer sub-sequences and constructing a non-redundant de Bruijn graph using a sliding window, where nodes with k−1 identical bases are connected, and the frequency between nodes is recorded as the weight of the edge;
deleting nodes connected by edges with weights below a set threshold to reduce the number of judgments in subsequent path selection;
selecting paths based on node information in the non-redundant de Bruijn graph and the consensus sequence; and
recording obtained sequences after path selection as reconstructed sequences.

20. A non-transitory computer-readable storage medium, having a computer program stored thereon, wherein the program, when executed by a processor, implements the method comprising:
obtaining Levenshtein distances between DNA sequences, and group DNA sequences with a Levenshtein distance less than or equal to a set threshold into the same clustering subgraph, thereby dividing multiple DNA sequences into multiple clustering subgraphs;
treating each DNA sequence in the clustering subgraph as a node, connecting nodes with the smallest Levenshtein distance between DNA sequences, and identifying the node with the highest degree as the backbone sequence of the clustering subgraph;
using the backbone sequence as a template sequence for sequencing data alignment, and constructing a beam search graph for other DNA sequences based on the relative positions of bases in the backbone sequence;
performing error correction and selecting an optimal path in the beam search graph using a beam search algorithm, and performing multiple sequence alignment on the DNA sequences involved in the optimal path to obtain a consensus sequence;
segmenting the DNA sequences of each clustering subgraph into k-mer sub-sequences and constructing a non-redundant de Bruijn graph using a sliding window, where nodes with k−1 identical bases are connected, and the frequency between nodes is recorded as the weight of the edge;
deleting nodes connected by edges with weights below a set threshold to reduce the number of judgments in subsequent path selection;
selecting paths based on node information in the non-redundant de Bruijn graph and the consensus sequence; and
recording obtained sequences after path selection as reconstructed sequences.
